(19) United States
(12) Patent Application Publication
(10) Pub. No.: US 2021/0077451 A1
(43) Pub. Date: Mar. 18, 2021

(54) A STABLE ORAL PHARMACEUTICAL COMPOSITION OF FERRIC CITRATE

(71) Applicant: KASHIV BIOSCIENCES, LLC, Bridgewater, NJ (US)

(72) Inventors: Parva Yogeshchandra PUROHIT, Ahmedabad (IN); Paras Rasiklal VASANANI, Ahmedabad (IN); Ashokkumar Hajabhai BARIA, Keshod (IN); Hinal Nitinkumar PATEL, Ahmedabad (IN); Krunal Ashokbhai GOYANI, Surat (IN)

(21) Appl. No.: 16/959,650

(22) PCT Filed: Dec. 31, 2018
(86) PCT No.: PCT/IB2018/060737

§ 371 Date: Jul. 1, 2020

(30) Foreign Application Priority Data
Jan. 2, 2018 (IN) IN201821000089

(51) Int. Cl. A61K 31/295; A61K 9/16; A61K 9/20; A61K 9/08; A61K 9/00

(52) U.S. Cl. CPC A61K 31/295; A61K 9/1652; A61K 9/1635; A61K 9/08; A61K 9/0095; A61K 9/2054

(57) ABSTRACT

The present invention relates to a pharmaceutical composition of ferric citrate or a pharmaceutically acceptable salt and one or more pharmaceutically acceptable excipients. The present invention specifically relates to oral pharmaceutical composition of ferric citrate and one or more pharmaceutically acceptable excipients. Further, the present invention relates to stable pharmaceutical composition of ferric citrate or a pharmaceutically acceptable salt which improves patient compliance by enhancing ease of administration of the above mentioned pharmaceutical composition. Moreover, the present invention describes the methods employed for manufacturing the above mentioned oral pharmaceutical composition of ferric citrate.

### TECHNICAL FIELD

The present invention relates to a stable oral liquid pharmaceutical composition (e.g. solution, suspension, syrup) comprising ferric citrate and a one or more pharmaceutically acceptable excipients.

The present invention further relates to a pharmaceutical granule composition comprising ferric citrate and a one or more pharmaceutically acceptable excipients.

The present invention also relates to a novel pharmaceutical sachet composition comprising an effective amount of ferric citrate, a flavouring agent, a sweetener and a one or more pharmaceutically acceptable excipients.

The present invention further relates to an orally disintegrating pharmaceutical composition comprising ferric citrate and a one or more pharmaceutically acceptable excipients.

The present invention further relates to a stable dispersible pharmaceutical composition comprising ferric citrate and a one or more pharmaceutically acceptable excipients.

The present invention further relates to a chewable pharmaceutical composition comprising ferric citrate and a one or more pharmaceutically acceptable excipients.

### BACKGROUND ART

Ferric iron containing compounds are useful in the treatment of a number of disorders, including, but not limited to, iron deficiency anemia, hyperphosphatemia and metabolic acidosis. Previous studies and inventions have reported the use of ferric compounds in binding with dietary phosphates, thereby provide the control over phosphate retention in patients suffering from renal failure and associated hyperphosphatemia (U.S. Pat. No. 5,753,706; 6,903,235; 8,093,423).

Elevated amounts of phosphate in the blood can be lowered by administering ferric iron containing compounds such as ferric citrate. Other studies and inventions have also reported use of ferric compounds in iron deficiency anemia (WO 2013/192565; WO 2016/141124).

WO 2011/011541 discloses ferric citrate tablets comprising 65 wt % to 92 wt % of ferric citrate and 4.5 wt % to 30 wt % binder, wherein at least 80% of the ferric citrate in the tablet is dissolved in a time less than or equal to 60 minutes. However, Production of such dosage forms containing ferric citrate in high content is associated with problems such as moldability during tableting, cracking, and difficulty in maintaining suitable hardness, affecting disintegration and dissolution properties of the dosage forms. To overcome such issues, WO 2012/005340 discloses ferric citrate tablets comprising polyvinyl alcohol-polyethylene glycol graft copolymer and polyvinyl alcohol-acrylic acid-methyl methacrylate copolymer and 70 wt % of ferric citrate.

In order to prepare dosage forms having properties (e.g., size) compatible with the patient as an end user, ferric citrate compositions available in market (Auryxia® marketed by Keryx Biopharmaceuticals) typically contain high percentages of the active ingredient relative to excipients. However, still total weight of tablet remains above 1 gm because approximately 1 gm ferric citrate is required to provide 210 equivalent iron per tablet. In addition to this, treatment of Hyperphosphatemia in Chronic Kidney Disease patients on Dialysis and treatment of Iron Deficiency Anemia in Chronic Kidney Disease patients not on Dialysis require a maximum of 12 tablets daily, i.e. maximum of 4 tablets three times a day. Such a high dosing frequency and dosage form properties (e.g. size) leads to problems related to patient compliance.

A need in the art exists for ferric citrate compositions that solve aforementioned problems and also incorporate therapeutically effective doses of the ferric citrate to effectively prevent and/or treat hyperphosphatemia, iron deficiency anemia and metabolic acidosis.

Moreover, Pharmaceutical compositions of ferric citrate are readily unstable when exposed to light and also in presence of moisture. Carbon dioxide is evolved, acetone is formed, and the ferric (FeIII) compounds are reduced. Ferric citrate complex are spherical in shape and the iron ions are tightly bound to bridging oxygen atoms, not citrate, and buried deep within the sphere. Citrate is believed to be located at the surface of the spheres, where it acts to stabilize the surface of the oxy-iron polymer, prevent additional cross-linking and cross complexation to ferric citrate complexes having molecular weights that greatly exceed 10,000 Daltons, and prevent precipitation of such complexes formed thereby at high pH. Once formed, high molecular weight ferric citrate complexes are very slow to dissociate to low molecular weight ferric citrate complexes that provide iron. A need in the art exists for compositions that overcome stability related problems associated with ferric citrate.

In the present invention, inventors have disclosed a novel approach for solving above mentioned problems by developing easy to swallow and stable pharmaceutical compositions such as liquid dosage form (solution, syrup, suspension), orally disintegrating tablet, dispersible tablet, chewable tablet, granules and sachet (powder or granules) of ferric citrate which provides better patient compliance in the treatment of patients with hyperphosphatemia in chronic kidney disease on dialysis and patients with iron deficiency anemia in chronic kidney disease not on dialysis.

### SUMMARY OF INVENTION

The present invention relates to a stable oral liquid pharmaceutical composition (e.g. solution, syrup, suspension) comprising ferric citrate and a one or more pharmaceutically acceptable excipients.

Further, the present invention relates to a pharmaceutical granules composition comprising ferric citrate and optionally a binder and one or more pharmaceutically acceptable excipients.

Further, the present invention relates a novel pharmaceutical sachet composition (powder or granules) comprising an effective amount of ferric citrate, a flavouring agent, a sweetener and one or more pharmaceutically acceptable excipients.

The present invention further relates to a stable orally disintegrating pharmaceutical composition comprising ferric citrate and a one or more pharmaceutically acceptable excipients.

Further, the present invention relates to a stable pharmaceutical dispersible composition of ferric citrate and a one or more pharmaceutically acceptable excipients.

Further, the present invention relates to a stable chewable composition of ferric citrate and one or more pharmaceutically acceptable excipients.

Moreover, the present invention also provides methods of manufacture of such compositions and methods of use thereof.

### DESCRIPTION OF EMBODIMENTS

The first aspect of the present invention relates to providing stable oral pharmaceutical compositions which provide greater ease of administration to patients who have difficulty in swallowing conventional ferric citrate tablets. The present invention relates to a stable oral pharmaceutical composition comprising ferric citrate and a process for preparing the pharmaceutical composition.

In second aspect of present invention, inventors have disclosed that cross linking in freshly prepared ferric citrate can be prevented or slowly reversed under certain conditions to impart stability to ferric citrate compositions. One embodiment of present invention covers stable pharmaceutical compositions of ferric citrate comprising ferric citrate and a stabilizer, wherein stabilizer is citric acid, malic acid, tannic acid, tartaric acid, phosphoric acid, acetic acid, lactic acid, maleic acid, and mixtures thereof. Another embodiment of present invention discloses stable pharmaceutical compositions of ferric citrate comprising ferric citrate and citric acid. Stabilizer used in present invention is from about 0.01% to about 50% of pharmaceutical composition. Amount of stabilizer disclosed in present invention is capable to supress and reverse ferric citrate cross complexation and maintain stability of ferric citrate over a period of many days.

In another aspect of present invention, inventors have disclosed taste masked formulations of ferric citrate to overcome metallic taste of ferric citrate compositions.

The term “active ingredient” refers to ferric citrate and related compounds formed as complexes between Fe (III) and citric acid in different molar ratios.

The inventors have defined term “stable” used here in the context of formulating pharmaceutical composition in which total impurities level of both specified and unspecified impurities complies as per ICH standards.

The term “liquid pharmaceutical composition” refers to solution or suspension or syrup of the ferric citrate alone or in combination with pharmaceutically acceptable ingredients in an aqueous liquid or a non-aqueous liquid.

The term “disintegrating composition” refers to a solid dosage form which disintegrates rapidly in the oral cavity of a patient, without chewing. The rate of disintegration can vary, but it is faster than the rate of disintegration of conventional solid dosage forms which are intended to be swallowed immediately after administration.

The term “dispersible composition” refers to solid dosage form that provides uniform dispersion for oral administration. Such Dispersible compositions comply with disintegration test wherein such tablets disintegrate within 3 minutes in water at 15-25° C. The dispersible tablet should comply with fineness of dispersion test in which 2 tablets are placed in 100 ml water and then stirred until completely dispersed.

The term “chewable tablet” refers to a tablet, regardless of its geometry, intended to be chewed by the patient and are not intended to be swallowed intact. Chewable formulations are intended to be used to provide a known dosage of active ingredient to children or patients who either will refuse to swallow an intact tablet or have difficulty doing so. Further, the act of chewing helps to break up the tablet particles as the tablet disintegrates and may increase the rate of absorption by the digestive tract.

The term “homogeneous dispersion” refers to the dispersion produced upon contact with water which ensures the uniformity of active ingredient for a reasonable period of time.

The term “sedimentation rate” refers to the rate at which the pharmacologically active ingredients settle from the dispersion.

The term “about” refers to any value which lies within the defined range by present inventors from a variation of up to ±10% of the claimed value.

The term “completely soluble” means solubility up to 100% and term “substantially soluble” means solubility up to at least 80%.

According to one embodiment of the present invention, stable liquid pharmaceutical composition comprising ferric citrate and one or more pharmaceutically acceptable excipients are disclosed. The present invention provides stable oral liquid pharmaceutical composition comprising ferric citrate and one or more pharmaceutically acceptable excipients in a liquid vehicle.

Oral solution of present invention comprises ferric citrate and one or more pharmaceutical excipient dissolved completely in pharmaceutical aqueous or non-aqueous media or combination thereof. Oral solution of present invention is ready to use or in form of concentrate which needs to be further diluted before use.

Oral suspension of present invention comprises ferric citrate, suspending agent and one or more pharmaceutical excipients dispersed completely or substantially in pharmaceutical aqueous or non-aqueous media.

Oral syrup of present invention comprises ferric citrate, one or more pharmaceutical excipients and 85% sucrose as a vehicle.

According to another embodiment of the present invention, there is provided a pharmaceutical granule composition of ferric citrate, binder and one or more pharmaceutically acceptable excipients. Further, the present invention relates to a pharmaceutical composition comprising granules comprising an inert core coated with ferric citrate and optionally a binder. The pharmaceutical granule composition of the present invention needs to be mixed with water before administration to the patient. Further, the pharmaceutical granule composition of the present invention dissolves or disperses completely or substantially in water.

According to another embodiment of the present invention, there is provided a pharmaceutical sachet composition comprising ferric citrate, a flavouring agent, a sweetener and one or more pharmaceutically acceptable excipients. The sachet composition of present invention is in the form of powder and or granule.

The sachet composition of present invention can be in the form of unit dose or multiple dose powder composition. Moreover, the sachet composition of present invention needs to be reconstituted with a diluent just before use. The resulting mixture has a pleasant mouth-feel and therefore can be conveniently administered to a patient as a drink. The drink can be a suspension or solution. Alternatively, the sachet formulations of the invention may be administered by a patient as a mixture with foods, such as mashed potatoes or oatmeal. This also affects the psychological state of the patient positively and also has higher compliance to the treatment.

According to another embodiment of the present invention, there is provided a stable orally dispersible pharmaceutical composition of ferric citrate. The present invention provides stable orally dispersible pharmaceutical composition of ferric citrate comprising ferric citrate and one or more pharmaceutically acceptable excipients wherein at least one excipient helps maintain uniform dispersion after dispersion in to vehicle. The stable orally dispersible pharmaceutical composition of present invention is present in the form of tablet, mini-tablet or pellets.

The dispersible composition of present invention rapidly disperses in water to produce a homogeneous dispersion to provide uniformity of content. The present invention particularly relates to the selective use of excipients that reduce the sedimentation rate which interact ionically with the active ingredient or which acts to reduce the surface tension between the aqueous media and the insoluble active or which increase viscosity of dispersion (viscosity building agents), thereby facilitating the maintenance of uniform dispersion of ferric citrate in the aqueous media.

According to another embodiment of the present invention, there is provided a stable chewable pharmaceutical composition of ferric citrate. The dispersible composition of the present invention comprises ferric citrate, taste masking agent and one or more pharmaceutically acceptable excipients.

According to another embodiment of the present invention, there is provided oral liquid pharmaceutical composition comprising ferric citrate and one or more pharmaceutically acceptable excipients selected from preservatives, sweetening agents, buffering agents, antioxidants, vehicle, flavoring agent and colouring agents.

According to another embodiment of the present invention, there is provided oral liquid pharmaceutical composition comprising ferric citrate and one or more pharmaceutically acceptable excipients selected from suspending agent, wetting agent, sweetening agents, buffering agents, anticaking agents, preservatives, stabilizer, antioxidants, vehicle, flavoring agent and coloring agents.

According to another embodiment of the present invention, there is provided oral solid pharmaceutical composition of ferric citrate and one or more pharmaceutically acceptable excipients selected from fillers, binders, super-disintegrants, polymers, lubricants, glidants, sweeteners, flavouring agents, sedimentation rate reducing agents (such as viscosity building agents, wetting agents etc.) coating agent, solvents and mixtures thereof.

According to another embodiment of the present invention, there is provided pharmaceutical solid composition of ferric citrate, wherein the ferric citrate is present in amount from about 100 mg to about 5000 mg, preferably from about 250 mg to about 4000 mg, more preferably from about 250 mg to about 3000 mg and from about 250 mg to about 2000 mg.

According to another embodiment of the present invention, there is provided pharmaceutical liquid composition of ferric citrate, wherein the ferric citrate is present in concentration from 10 mg/ml to 1500 mg/ml.

According to another embodiment of the present invention, there is provided a pharmaceutical liquid composition of ferric citrate, wherein final pH of liquid composition is less than 7.

The vehicle used in the pharmaceutical composition of the present invention is selected from the group consisting of water, glycerin, propylene glycol sorbitol solution, polyethylene glycol and the like. The vehicle is used either alone or in combination in the range of about 1% to about 95% weight by volume of the total liquid pharmaceutical composition.

The suspending agent used in the pharmaceutical composition of the present invention are selected from the group consisting of hypromellose, polyvinylpyrrolidone, magnesium aluminium silicate, xanthan gum, carbomer, hydrocolloid gums like guar gum, gum tragacanth and cellulose derivatives for example methyl-, ethyl- and propyl celluloses; hydroxyalkyl-celluloses, hydroxyl propyl celluloses, hydroxylpropylalkyl celluloses, sodium carboxy methyl cellulose, microresins, polyethylene glycol, polyethylene oxide, sodium alginate and the like. The suspending agents used either single or in combinations in the range of about 0.05% to about 20%, by weight by volume of the total liquid composition.

The preservatives used in the pharmaceutical composition of the present invention are selected from the group consisting of benzyl alcohol, methyl and propyl parabens, butyl paraben, ethyl paraben, sorbic acid, potassium sorbate, benzalkonium chloride, benzoic acid and its derivatives such as sodium benzoate and the like. The preservatives used either single or in combinations in the range of about 0.005% to about 20%, by weight by volume of the total liquid composition.

The buffers to be used in the present invention to maintain the pH below 7 can be conventional buffers of organic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium gluconate mixture, gluconic acid-sodium hydroxide mixture, gluconic acid-potassium gluconate mixture, etc.), oxalate buffers (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). The buffering agents used either single or in combinations to adjust pH (less than 7.0) in the range of about 0.01% to about 10% weight by volume of the total liquid composition.

The anticaking agents used in the pharmaceutical composition of the present invention are selected from the group consisting of colloidal silicon dioxide alcium phosphate tribasic, magnesium oxide, magnesium silicate, calcium silicate and the like. The anticaking agent used either single or in combinations in the range of about 0.01% to about 20% weight by volume of the total liquid composition.

The stabilizers used in the pharmaceutical composition of the present invention are selected from the group consisting of citric acid, malic acid, tannic acid, tartaric acid, phosphoric acid, acetic acid, lactic acid, maleic acid, and mixtures thereof. The stabilizers is used either single or in combinations in the range of about 0.01% to about 45% weight by volume of the total liquid pharmaceutical composition.

The antioxidants used in the pharmaceutical composition of the present invention are selected from the group consisting of sodium metabisulfite, ascorbic acid, sodium sulfite, sodium bisulfate, sodium thiosulfate, sodium ascorbate, sodium formaldehydesulfoxylate, malic acid, alkyl gallates like propyl gallate, lauryl gallate, or octyl gallate and the like. The antioxidant used either single or in combinations in the range of about 0.01% to about 20% weight by volume of the total liquid composition.

The fillers used in the pharmaceutical composition of the present invention are selected from the group consisting of a lactose, microcrystalline cellulose, starch, pre-gelatinized starch, calcium phosphate, calcium sulfate, calcium carbonate, mannitol, sorbitol, xylitol, sucrose, maltose, fructose, dextrose, maltodextrin, dextrates, dextrin, and the like thereof. The fillers may present in an amount from about 1% to 80% weight by weight of the composition.

The binders used in the pharmaceutical composition of the present invention are selected from the group consisting of a starches, natural and synthetic gums, cellulose derivatives, gelatin, povidone, copovidone, polyethylene glycol, waxes, sodium alginate, alcohols, water, and the like thereof. The binders may present in an amount from about 0.01% to 20% weight by weight of composition.

The superdisintegrants used in the pharmaceutical composition of the present invention are selected from the group consisting of a crospovidone (crosslinked PVP), sodium starch glycolate, crosslinked sodium carboxymethyl cellulose (croscarmellose sodium), low substituted hydroxypropyl cellulose, and mixtures thereof. According to the present invention, preferable disintegrants are crospovidone and sodium starch glycolate. The disintegrants may present in an amount from about 1% to 30% weight by weight of composition.

The polymers used in present invention are selected from the group consisting of hypromellose, copovidone, povidone, methyl cellulose, hydroxyethyl cellulose, hydroxypropyl cellulose, ethylcellulose, pyroxylin, polyethylene oxide, polyvinyl alcohol, polyethylene glycol, and polyvinyl caprolactam-polyvinyl acetate-polyethylene glycol, polysaccharides, polypeptides, and methacrylic acid copolymers, ethyl acrylic acid copolymers. A preferred polymer is acrylic products such as poly(meth)acrylate (EUDRAGIT™) copolymers are available in various physical forms, for example, EUDRAGIT EPO being a powder form of EUDRAGIT E 100. The polymers may present in an amount from about 1% to about 60% weight by weight of composition.

The lubricants used in the pharmaceutical composition of the present invention are selected from the group consisting of a calcium stearate, Glyceryl monostearate, Glyceryl palmitostearate, hydrogenated castor oil, hydrogenated vegetable oil, light mineral oil, magnesium stearate, mineral oil, polyethylene glycol, stearic acid, zinc stearate, and sodium stearyl fumarate and a combination thereof. A preferred lubricant is magnesium stearate and sodium stearyl fumarate and may present in amount from about 0.1% to 10% weight by weight of composition.

The glidants used in the pharmaceutical composition of the present invention are selected from the group consisting of a starch, talc, lactose, stearates, dibasic calcium phosphate, magnesium carbonate, magnesium oxide, calcium silicate, and colloidal silicon dioxide (Aerosil) and the like thereof. The Glidants may present in an amount from about 0.1% to 20% weight by weight of composition.

The pharmaceutical composition of present invention may also contain a chelating agent to protect the active agent either during storage or when in use. Examples of chelating agents include, for example, polyacrylic acid, citric acid, edetic acid, disodium edetic acid, and the like. The chelating agent may present in an amount from about 0.1% to 20% weight by weight of composition.

The taste masking agent used in the pharmaceutical composition of the present invention includes sweeteners, flavouring agents, β-Cyclodextrin, resins and like thereof and combination thereof.

The sweeteners used in the pharmaceutical composition of the present invention are selected from the group consisting of a alitame, acesulfame potassium, aspartame, sodium cyclamate, D-tryptophan, dextrose, erythritol, fructose, galactose, glycerol, glycyrrhizin, glucose, isomalt, xylitol, xylose, lactitol, lactose, levulose, mannitol, maltitol, maltodextrin, maltol, maltose, corn syrup, neohesperidin dihydrochalcone, neotame, sodium saccharin, siclamate, sorbitol, sucralose, sucrose, tagatose, taumatin, trehalose, and the like thereof. The amount of sweetening agent may vary from 0.1 to 20%, preferably from 0.2 to 10% weight by weight of composition.

The flavouring agents used in the pharmaceutical composition of the present invention are selected from the illustrative list of such agents includes volatile oils, synthetic flavour oils, flavouring aromatics, oils, liquids, oleoresins or extracts derived from plants, leaves, flowers, fruits, stems and combinations thereof. A non-limiting representative list of examples includes spearmint oil, cinnamon oil, peppermint oil, clove oil, bay oil, thyme oil, cedar leaf oil, anethole, lemon, linalool, menthol, eucalyptol, orange, menthol, citrus oil, mints, vanilla, orange, cinnamon, tartaric acid, thymol, vanilla, Tutti Frutti, banana, strawberry, cherry, chocolate aroma or peppermint aroma and the like thereof. The amount of flavouring agent may vary from 1 to 20%, preferably from 2 to 10% weight by weight of composition.

The excipients used in the pharmaceutical composition of the present invention, which reduce the sedimentation rate of the active ingredient, may include polymers, waxes, wetting agents or others that interact ionically with the active ingredient. The wetting agent acts by reducing the surface tension between the aqueous media and the insoluble active, thereby facilitating the active's maintenance in the aqueous media. The viscosity-building agents may be selected from but not limited to xanthan gum, carrageenan, tragacanth, guar gum, pectin, carboxymethylcellulose, hydroxypropyl methylcellulose, hydroxypropylcellulose, methylcellulose, microcrystalline cellulose and carboxymethylcellulose sodium blends, and mixtures thereof. The viscosity-building agent provides both body and mouth feel to the preparation. The viscosity-building agent must be selected carefully to ensure compatibility with the drug and the other components of the formulations. The hydrophilic polymer may present in an amount from about 2% to 75% by weight of composition, preferably from about 10% to 70% weight by weight of composition, and most preferably from about 5% to 50% weight by weight of composition.

The wetting agents used in the pharmaceutical composition of the present invention are selected from the surfactants, including nonionic, cationic, anionic, and zwitterionic type surfactants. These include sorbitan fatty acid esters, polyoxyethylene sorbitan fatty acid esters (Tween®), polyoxyethylene fatty acid esters, poly-oxyethylene-polyoxypropylene copolymers and block copolymers and the like thereof. The wetting agents may present in an amount in a range of from about 1 to about 60%, preferably from about 10% to about 40%, and is more preferably employed in an amount in a range of from about 20% to about 30% weight by weight of composition.

The inert core used in pharmaceutical granule compositions described herein can be comprised of any pharmaceutically inert compound, e.g., a filler. The inert core onto which the ferric citrate and optional binder is applied is usually comprised of sugars and starch (e.g. nonpareil seeds) or cellulosic materials, ionic compositions (e.g. calcium dibasic phosphate) or combinations thereof, for example sugar derivatives such as lactose, sucrose, hydrolyzed starch (maltodextrins) or celluloses or mixtures thereof. A currently preferred excipient for the inert core is microcrystalline cellulose (AVICEL®). Any pharmaceutically acceptable AVICEL® grade can be used in the context of the invention, e.g., AVICEL® 101, AVICEL® 102 and the like.

Co-solvents used in the pharmaceutical composition of present invention are selected from the group consisting of a propylene glycol, glycerin, water soluble polyethylene glycol (PEG) polymers, propylene glycol and a like thereof. The solvent/co-solvent is present in an amount ranging from about 1% to about 30% weight by weight of composition, preferably from about 5% to about 20% weight by weight of composition.

The colouring agents used in the pharmaceutical composition of the present invention are selected from the D&C Yellow No. 10 natural or synthetic dyes but not restricted to carmoisine, FD&C Yellow No. 5, FD&C Yellow No. 6, FD&C Red No. 3, FD&C Red No. 20, FD&C Blue No. 2, D&C Green No. 5, D&C Yellow No. 7, D&C Orange No. 5, D&C Red No. 8, caramel and the like. The coloring agents used either single or in combinations in the range of about 0.001% to about 2% weight by volume of the total liquid composition.

Examples of film-forming agents include, but are not limited to, cellulose derivatives such as methylcellulose, hydroxymethyl cellulose, hydroxyethyl cellulose, hydroxypropyl cellulose, hydroxymethyl ethylcellulose, hydroxypropyl methylcellulose, sodium carboxymethyl cellulose, and ethyl cellulose; waxes; fat substances; or mixtures thereof. Alternatively, commercially available coating compositions comprising film forming polymers marketed under various trade names, such as Opadry®, may be used for coating. Examples of solvents used for preparing the coating solution as well as granulating solution are selected from methyl alcohol, ethyl alcohol, isopropyl alcohol, n-butyl alcohol, acetone, acetonitrile, chloroform, methylene chloride, water, or mixtures thereof.

The composition of the present invention may be in the form of orally disintegrating tablets, orally dispersible tablets, granules, pellets, minitablets, sachets, chewable tablets, powder for reconstitution, or a like thereof which can easily disintegrate after administration or dispersed/reconstituted in a suitable medium during administration, Wherein, granules, pellets, minitablets are further coated with suitable coating agent.

The pharmaceutical composition of the present invention may further be film-coated using techniques well known in the art such as spray coating in a conventional coating pan or a fluidized bed processor or dip coating. Alternatively, coating may also be performed using the hot melt technique. The film coat comprises film-forming polymers, one or more pharmaceutically acceptable excipients and pharmaceutically acceptable solvents.

The pharmaceutical composition of the present invention can be obtained by a known conventional methods like simple mixing or homogenization or milling, dry granulation, wet granulation, direct compression, roller compaction, fluidized bed granulation, rapid mixture granulation, spray drying, freeze drying, solvent evaporation, hot-melt extrusion, extrusion spheronization, melt granulation or a like that.

The pharmaceutical composition of the present invention can be used in the treatment of patients with hyperphosphatemia in chronic kidney disease on dialysis and iron deficiency anemia in chronic kidney disease not on dialysis.

The present invention is illustrated below by reference to the following examples. However, one skilled in the art will appreciate that the specific methods and results discussed are merely illustrative of the invention, and not to be construed as limiting the invention, as many variations thereof are possible without departing from the spirit and scope of the invention.

### Example 1 Granule Composition of Ferric Citrate

TABLE 1

| Ingredients | Amount | | Percentage |
|---|---|---|---|
| Milled Ferric Citrate | 1190.7 | mg | 75.0% |
| Copovidone (Kollidon VA64) | 258.8 | mg | 16.3% |
| Crospovidone (Polyplasdone | 56.3 | mg | 3.5 |
| XL) | | | |
| Hydroxypropyl cellulose | 64.25 | mg | 4.1% |
| Magnessium stearate | 17.3 | mg | 1.1% |
| Total | 1323 | mg | 100% |
| Coating composition | | | |
| Hypromellose Opadry | | | 12% |
| Titanium dioxide | | | 3% |
| FD&C Yellow | | | q.s. |
| Water | | | 100% |

Procedure:

1) In a GMX high shear granulator, required quantity of milled ferric citrate, Copovidone (Kollidon VA64), Crospovidone (Polyplasdone XL), Hydroxypropyl cellulose were mixed for 2 minutes at 500 rpm.

2) Deionized water was added at an approximate rate of 18 g/min over 10 minutes, while mixing at a rate of 900 rpm with a chopper speed of 1500 rpm. The granules were dried in fluid bed drier for 5-8 minutes at an inlet temperature of 65° C.

3) The granules were screened through a 16 mesh hand-screen, then through a 25 mesh hand screen to remove over-sized granules and clumps. The magnesium stearate was screened through a 25 mesh hand-screen.

4) Granules and magnesium stearate were blended for 2 minutes in a 2 quart v-blender. Granules thus prepared were.

5) An aqueous film coating suspension of hypromellose (opadry), titanium dioxide and FD&C Yellow was prepared in a stainless steel kettle and mixer. The granules were charged into a fully perforated pan coater, and the coating suspension was sprayed onto the cascading product bed. The theoretical weight gain for the subject batch was 3%.

6) Upon completion of the spraying step the granules were dried. Film coated granules were discharged into intermediate bulk containers. The film coated granules were packaged in HPDE bottles with desiccant or sachet.

### Example 2 Oral Dispersible Tablet of Ferric Citrate

TABLE 2

| Ingredients | Amount | |
|---|---|---|
| Ferric Citrate | 1190 | mg |
| Hydroxypropyl cellulose | 90 | mg |
| MCC | 195 | mg |
| Croscarmellose sodium | 80 | mg |
| Tutti Frutti | 25 | mg |
| Aspartame | 20 | mg |
| Acesulfame potassium | 20 | mg |
| Magnesium stearate | 14 | mg |
| Total | 1634 | mg |

Procedure:

1) Milled ferric citrate, hydroxypropyl cellulose, microcrystalline cellulose, and croscarmellose sodium were mixed for 2 minutes at 500 rpm in a high shear granulator 4 L bowl.

2) Deionized water was added at an approximate rate of 18 g/min over 10 minutes, while mixing at a rate of 900 rpm with a chopper speed of 1500 rpm.

3) The granules were dried in an fluid bed for 5-8 minutes at an inlet temperature of 65° C. The moisture content after drying was measured.

4) The granules were screened through a 16 mesh hand-screen, then through a 25 mesh hand screen to remove over-sized granules and clumps and then mixed with tutti frutti, aspartame & acesulfame potassium in a blander for 3 minutes & keeping the blend ready for lubrication further.

5) Blend and magnesium stearate were blended for 2 minutes in a v-blender. Tableting was performed by using a suitable tablet press.

### Example 3 Oral Solution of Ferric Citrate

TABLE 3

| Ingredient | Amount |
|---|---|
| Ferric Citrate | 20.0% |
| Citric acid | 4.6% |
| Mannitol | 5.0% |
| Cherry flavour | 0.045% |
| D&C Red No.8 | 0.05% |
| Citrate buffer | 1% |
| Water q.s. | 100% |

Procedure:

1) Milled ferric citrate and Citric acid were dissolved in sufficient quantity of deionized water by continuous stirring.

2) Separately Mannitol, Cherry flavour and D&C Red No. 8, Citrate buffer were also dissolved in sufficient quantity of deionized water by continuous stirring.

3) Both the solutions were mixed and final volume was made up with water up to 100 ml.

1-12. (canceled)

13. A liquid oral pharmaceutical composition comprising a pharmaceutically acceptable ferric salt, stabilizer and one or more pharmaceutically acceptable excipients, wherein the composition is stabilized using one or more stabilizers.

14. The liquid pharmaceutical composition according to claim 13, wherein the pharmaceutical composition is present in form of solution, suspension, syrup or a like thereof.

15. The liquid pharmaceutical composition according to claim 13, wherein one more pharmaceutically acceptable excipients are selected from the group consisting of stabilizers, buffering agent, taste masking agent, fillers, suspending agent, wetting agents, viscosity-building agents, antioxidants, vehicle, flavouring agent, preservatives, colouring agents or combination thereof.

16. The liquid pharmaceutical composition according to claim 15, wherein the stabilizer is selected from the group consisting of citric acid, malic acid, tannic acid, tartaric acid, phosphoric acid, acetic acid, lactic acid, maleic acid, and mixtures thereof.

17. The liquid pharmaceutical composition according to claim 15, wherein the buffering agent is selected from the group consisting of citrate buffers, succinate buffers, tartrate buffers, fumarate buffers, gluconate buffers, oxalate buffers, lactate buffers, acetate buffers, and mixtures thereof.

18. The liquid pharmaceutical composition according to claim 15, wherein the taste masking agent is selected from the group consisting of sweeteners, flavouring agents, β-Cyclodextrin, resins and mixtures thereof.

19. The liquid pharmaceutical composition according to claim 15, wherein the filler is selected from the group consisting of lactose, microcrystalline cellulose, starch, pre-gelatinized starch, calcium phosphate, calcium sulfate, calcium carbonate, mannitol, sorbitol, xylitol, sucrose, maltose, fructose, dextrose, maltodextrin, dextrates, dextrin, and mixture thereof.

20. The liquid pharmaceutical composition according to claim 15, wherein the wetting agent is selected from the group consisting of sorbitan fatty acid esters, polyoxyethylene sorbitan fatty acid esters (Tween®), polyoxyethylene fatty acid esters, polyoxyethylene-polyoxypropylene copolymers, block copolymers and mixtures thereof.

21. The liquid pharmaceutical composition according to claim 15, wherein the stabilizer is present in an amount ranging from about 0.01% by weight to about 50% by weight of composition.

22. The liquid pharmaceutical composition according to claim 13, wherein the pharmaceutically acceptable ferric salt is present in an amount ranging from about 1 to about 95% by weight of composition.

23. The liquid pharmaceutical composition according to claim 13, wherein the pharmaceutically acceptable ferric salt is ferric citrate.

24. The liquid pharmaceutical composition according to claim 23, wherein the ferric citrate is present in dose ranging from about 0.5 gm to about 15 gm.

25. The liquid pharmaceutical composition according to claim 13, wherein pH of the liquid pharmaceutical composition is less than 7.

26. The liquid pharmaceutical composition according to claim 13, wherein the composition is administered to the subjects for the treatment of hyperphosphatemia and/or anemia.

27. The liquid pharmaceutical composition according to claim 13, wherein the composition is administered to the subject suffering from chronic kidney disease.
